(19) United States
(12) Patent Application Publication
(10) Pub. No.: US 2006/0110732 A1
(43) Pub. Date: May 25, 2006

(54) Method for the identification of ligands

(76) Inventors: Roger F. Bone, Bridgewater, NJ (US); Dionisios Rentzeperis, Downingtown, PA (US); Hossein Askari, West Chester, PA (US); Barry A. Springer, Wilmington, DE (US)

Correspondence Address: WILSON SONSINI GOODRICH & ROSATI, 650 PAGE MILL ROAD, PALO ALTO, CA, 94304-1050, US

(21) Appl. No.: 10/519,757

(22) PCT Filed: Jul. 23, 2003
(86) PCT No.: PCT/US03/23247

§ 371 Date: Oct. 4, 2005

Related U.S. Application Data
(60) Provisional application No. 60/398,023, filed on Jul. 24, 2002.
(60) Provisional application No. 60/413,843, filed on Sep. 27, 2002.
(60) Provisional application No. 60/413,866, filed on Sep. 27, 2002.

(51) Int. Cl. C12Q 1/68; G01N 33/53

(52) U.S. Cl. 435/6; 435/7.1

(57) ABSTRACT

The present invention relates generally to a method of identifying ligands that modulate protein-protein interactions. More particularly, the present invention relates to methods of determining agonists or antagonists of a co-regulator dependent target molecule based on the ability to modify the stability of the target molecule in a tissue-selective manner.

This application claims priority benefit to U.S. Provisional Application Nos. 60/398,023 filed Jul. 24, 2002, and 60/413,866 and 60/413,843, both filed Sep. 27, 2002, which are incorporated by reference herein in their entirety.

### FIELD OF THE INVENTION

The present invention relates generally to a method of identifying ligands for protein-protein interactions whose, affinity is modulated by ligands or allosteric regulators. More particularly, the present invention relates to methods of determining the tissue selectivity of a ligand for a co-regulator dependent target molecule based on the ability of the ligand modify the stability of the receptor when in the presence of the co-regulator.

### BACKGROUND OF THE INVENTION

A central theme in signal transduction and gene expression is the constitutive or inducible interaction of protein-protein modular domains. Knowledge of ligands that can potentiate these interactions will provide information on the nature of the molecular mechanisms underlying biological events and on the development of therapeutic approaches for the treatment of disease. Existing methods for the identification of ligands are cumbersome and limited particularly in the case of proteins of unknown function.

Nuclear receptors are members of a superfamily of transcription factors controlling cellular functions including reproduction, growth differentiation, and lipid and sugar homeostasis. Their function is regulated by a diverse set of ligands (xenobiotics, hormones, lipids and other known and undiscovered ligands). To date 48 nuclear receptors have been identified, 28 of which are known ligands with the remaining 20 classified as orphans. The biology of the receptors is complex and tissue specific (Shang & Brown, Science 295:2465-2468, 2002) and the molecular mechanism of action appears to be a function of preferential recruitment of accessory proteins, referred as co-regulators, that modulate the function of these receptors in a ligand independent or dependent fashion. Recruitment of the appropriate co-regulator can result in gene transcription or repression.

Panvera offers reagents for the discrimination of agonist from antagonist ligands for the estrogen receptor subtype beta and has presented publicly data on the preferential recruitment of co-activator proteins. See, e.g, Bolger et al., Environmental Health Perspectives 106:1-7 (1998) and Panvera corporate presentation presented at the Orphan Receptor Meeting San Diego (June 2002). Panvera's reagents are used in assays based on fluorescence resonance energy transfer (FRET).

There are publications on similar assays for other nuclear receptors (ER-α, the ERR and PPAR family) that are also based on FRET. See, e.g., Zhou et al., Molecular Endocrinology 12:1594-1604 (1998) and Coward et al., 98:8880-8884, (2001). And similar experiments have been done using Biacore technology. See, e.g., Cheskis et al., J. Biological Chemistry 11384-11391 (1997) and Wong et al.; Biochemistry 40:6756-6765 (2001).

Cellular assays exist where the readout is gene expression. See, e.g., Camp et al., Diabetes 49:539-547 (2001) and Kraichely et al., Endocrinology, 141:3534-3545, (2000). For example, Karo-Bio has developed a gene expression readout assay to include conformational sensitive peptide probes for discrimination of agonist from antagonist ligands for nuclear hormone receptors. See, e.g., Paige et al., PNAS 96:3999-4004 (1999) and Presentation by Karo-Bio at the Orphan Receptor Meeting, San Diego (June 2002).

Greenfield et al., Biochemistry 40:6446-6652 (2001) reports the thermal stablization of the ER-α receptor in the presence of estradiol. However, the reference does not teach the identification of a molecule as an agonist or an antagonist of the ER-α receptor.

The art discussed above suffers from several drawbacks. For example, in the analysis of nuclear receptors, gene expression readout assays and cell based assays, counter-screens are required to validate that ligands or co-regulators identified interact directly with the receptor of interest and not through other proteins that can produce a signal transduction or gene activation/repression assay readout. In addition, cell readout technology lacks the sensitivity in identifying weak ligands (typically compounds of affinities of greater than 1 μM are rarely identified), and is only applicable to compounds that have a good cell permeability profile. Other commercial in vitro assays require the knowledge of ligands for establishing competitive displacement assays, or the use of them as tools to validate FRET based co-regulator assays.

Thus, there is a need for an accurate, reliable technology that facilitates the rapid, high-throughput identification of ligands for co-regulator dependent receptors and further identification of their effect on the receptor when in the presence of a co-regulator, particularly in a tissue-selective or gene-selective manner.

### SUMMARY OF THE INVENTION

The present invention meets one or more of these needs. The present invention provides a method of determining the tissue selectivity of a ligand for a co-regulator dependent target molecule. The method comprises providing a set of ligands that modify the stability of the target molecule and screening one or more ligands of the set for their ability to further modify the stability of the target molecule in the presence of one or more tissue-selective co-regulators for the target molecule. A further modification of stability of the target molecule in the presence of a ligand of the set and a co-regulator indicates whether the ligand is an agonist or an antagonist of the target molecule when in the presence of the tissue-selective co-regulator, thereby determining the tissue selectivity of the ligand for the target molecule.

The invention provides another method of determining the tissue selectivity of a ligand for a co-regulator dependent target molecule. The method comprises providing a set of ligands that shift the thermal unfolding curve of the target molecule and screening one or more ligands of the set for their ability to further shift the thermal unfolding curve of the target molecule in the presence of one or more tissue-selective co-regulators for the target molecule. A further shift in the thermal unfolding curve of the target molecule in the presence of a ligand of the set and a co-regulator indicates whether the ligand is an agonist or an antagonist of the target molecule when in the presence of the tissue-selective co-regulator, thereby determining the tissue selectivity of the ligand for the target molecule.

An advantage of the methods of the present invention is that neither gene expression readout and cell based assays, nor the use of known ligands to establish the assay are required. The ability to generate information in such a direct fashion allows the discovery of drugs with desired properties, to test therapeutic hypotheses and decrypt orphan receptors.

By use of isolated and/or purified proteins and peptides in a single unifying assay, one can identify ligands that are involved in modulating protein-protein interactions and predict biological response. Not only can ligands be identified, but also the intrinsic affinity for the target protein can be calculated which then can be used to correlate to biological activity. The information generated can also be used to predict or determine the pharmacology and tissue specificity of drugs and to identify ligands for orphan receptors that in turn can be used as tools to deconvolute the biology of these proteins to test therapeutic hypotheses. More specifically, the invention provides for tissue-selective drug lead discovery, for agonists and antagonists depending upon the tissue of interest, along with gene-selective drug lead discovery.

Data generated by methods of the present invention does not require counter-screening, as changes in the melting temperature of a target molecule, such as a protein is a direct consequence of the thermodynamic linkage of the binding energy of macromolecules and ligands to the protein of interest Further, affinities of a ligand to a target molecule are more sensitive (affinities of pM to mM are determined). Further, the present invention is not limited by compounds with poor cell permeability. Also, as mentioned above, the present invention does not require known ligands to establish an assay, making it extremely powerful for deconvoluting orphan receptors.

Further features and advantages of the present invention are described in detail below with reference to the accompanying drawings.

### BRIEF DESCRIPTION OF THE DRAWINGS/FIGURES

FIG. 1 illustrates experimental results expected for the identification of an agonist ligand in the presence of a co-activator.

FIG. 2 illustrates experimental results expected for the identification of an antagonist ligand in the presence of a co-activator.

FIG. 3 illustrates binding constants, Ka, for co-activator proteins SRC-1, SRC-2 and SRC-3 in the presence of ER-α ligands.

FIG. 4 illustrates binding constants, Ka, for co-activator proteins SRC-1, SRC-2 and SRC-3 in the presence of ER-β ligands.

FIG. 5 illustrates experimental results expected for the identification of an partial agonist

FIG. 6A illustrates calculated binding constants for the co-activator peptide SRC-2-NR2 in the absence and in the presence of PPAR-γ ligands.

FIG. 6B illustrates calculated binding constants for the co-repressor peptide NCoR-1 in the absence and in the presence of PPAR-γ ligands.

FIG. 6C illustrates the ratio of the calculated affinities for the co-activator and co-repressor peptides from FIGS. 6A and 6B.

### DETAILED DESCRIPTION OF THE PREFERRED EMBODIMENTS

In the following description, reference will be made to various terms and methodologies known to those of skill in the biochemical and pharmacological arts. Publications and other materials setting forth such known terms and methodologies are incorporated herein by reference in their entireties as though set forth in full.

In embodiments of the present invention, methods are provided for the determination of the tissue selectivity of a ligand for co-regulator dependent target molecules, which are capable of unfolding, based upon molecules that modify the stability of the target molecule. Ligands that modify the stability of the target molecule can be screened in the presence of the target molecule and one or more tissue-selective co-regulators for their ability to further modify the stability of the target molecule. Whether the stability of the target molecule is further modified is an indication as to whether the ligand is an agonist or an antagonist of the target molecule when in the presence of the tissue-specific co-regulator. Based upon this information, the tissue-selectivity of a ligand for a target molecule can be determined.

In other embodiments of the invention, methods are provided for the determination of the tissue selectivity of a ligand for co-regulator dependent target molecules which involve the unfolding of a target molecule due to a thermal change. Ligands that shift the thermal unfolding curve of the target molecule can be screened in the presence of the target molecule and one or more tissue-selective co-regulators for their ability to further shift the thermal unfolding curve of the target molecule. Whether the thermal unfolding curve of the target molecule is further shifted is an indication as to whether the ligand is an agonist or an antagonist of the target molecule when in the presence of the tissue specific co-regulator. Based upon this information, the tissue-selectivity of a ligand for a target molecule can be determined.

The terms “tissue specificity” or “tissue selectivity” of a ligand for a target molecule refer generally to the effect that a ligand has on a target molecule in a particular tissue such as, e.g., whether the ligand acts as an agonist or an antagonist for a target molecule in the particular tissue. The effect of the ligand on the target molecule may be controlled by, e.g., the identity, nature, and levels of co-regulators that are expressed by are otherwise present in the tissue of interest.

The term “target molecule” encompasses peptides, proteins, nucleic acids, and other receptors. The term encompasses both enzymes and proteins which are not enzymes. The term encompasses monomeric and multimeric proteins. Multimeric proteins may be homomeric or heteromeric. The term encompasses nucleic acids comprising at least two nucleotides, such as oligonucleotides. Nucleic acids can be single-stranded, double-stranded or triple-stranded. The term encompasses a nucleic acid which is a synthetic oligonucleotide, a portion of a recombinant DNA molecule, or a portion of chromosomal DNA.

The term “target molecule” also encompasses portions of peptides, proteins, and other receptors which are capable of acquiring secondary, tertiary, or quaternary structure through folding, coiling or twisting.

The target molecule may be substituted with substituents including, but not limited to, cofactors, coenzymes, prosthetic groups, lipids, oligosaccharides, or phosphate groups. The term “target molecule” and “receptor” are synonymous.

More specifically, the target molecules utilized in the present invention are co-regulator dependent. By “co-regulator dependent” it is meant that the target molecule is capable of binding at least one ligand and binding at least one co-regulator. Further, the activity of the target molecule, whether in a ligand dependent or independent function, is dependent upon, at least in part, by a co-regulator. Co-regulator dependent target molecules include, but are not limited to, nuclear receptors.

Nuclear receptors, and the role of co-regulators relating thereto, are described in Aranda and Pascual, Physiological Reviews 81:1269-1304 (2001); Collingwood et al, Journal of Molecular Endocrinology 23:255-275 (1999); Robyr et al., Molecular Endocrinology 23:329-347 (2000); and Lee et al., Cellular and Molecular Life Sciences 58:289-297 (2001), the references incorporated by reference herein by their entireties.

Further, the co-regulator dependent target molecules encompass vertebrate species, including, but not limited to humans, as well as invertebrates, including but not limited to insects.

Illustratively, insects contain hundreds of nuclear receptors, for which ligands can be identified as agonists or antagonists. See Laudet, J. Molecular Endocrinology 19:207-226 (1997) and Maglich et al., Genome Biology 2:1-7 (2001) for a discussion of nuclear receptors present in vertebrates, nematodes and arthropods, the references incorporated by reference herein by their entireties.

The term “protein” encompasses full length or polypeptide fragments. The term “peptide” refers to protein fragments, synthetic or those derived from peptide libraries. As used herein, the terms “protein” and “polypeptide” are synonymous.

The term “co-regulator” refers to chemical compounds of any structure, including, but not limited to nucleic acids, such as DNA and RNA, and peptides that modulate the target molecule in a ligand dependent or independent fashion. The term refers to natural, synthetic and virtual molecules. More specifically, the term refers to a peptide or polypeptide/protein, natural or synthetic that modulates the target molecule in a ligand dependent or independent fashion. The term encompasses peptides that are derived from natural sequences or from phage display libraries. The peptide can be fragments of native proteins. More specifically, the term refers to co-activators and co-repressors.

The term “co-activator” refers to a molecule which binds to a target molecule and causes an activation of or an increase in an activity of the target molecule. In embodiments of the invention, the term refers to molecules that bind to a target molecule to induce gene transcription or to induce a signaling function (e.g. signal transduction).

The term “co-repressor” refers to a molecule which binds to a target molecule and causes a deactivation or a decrease in an activity of the target molecule. In embodiments of the invention, the term refers to molecules that bind to a target molecule to repress gene transcription or to repress a signaling function (e.g. signal transduction).

The term “agonist” refers to a molecule which binds to a target molecule and induces or recruits a co-activator for binding to the target molecule.

In embodiments of the invention, the term “agonist” refers to a molecule that binds to a nuclear receptor and recruits a co-activator. In these embodiments, the term more specifically refers to a molecule that alters gene expression by inducing conformational changes in a nuclear receptor that promote direct interactions with co-activators.

The term “antagonist” refers to a molecule which binds to a target molecule and induces or recruits a co-repressor for binding to the target molecule.

In embodiments of the invention, the term “antagonist” refers to a molecule that binds to a nuclear receptor and recruits a co-repressor. In these embodiments, the term more specifically refers to a molecule that alters gene expression by inducing conformational changes in a nuclear receptor that promote direct interactions with co-repressors.

The term “partial agonist” refers to a molecule which binds to a target molecule and has the ability to induce or recruit a co-activator and a co-repressor for binding to the target molecule. It should be understood that the term can include molecules which may recruit a co-activator more strongly than a co-repressor, molecules which may recruit a co-activator with about the same affinity as a co-repressor, and/or molecules which may recruit a co-repressor more strongly than a co-activator. The concept of partial agonism is further discussed below.

The term “ligand” refers to a compound which is tested for binding to the target molecule in the presence of or absence of additional compounds, such as co-regulators. This term encompasses chemical compounds of any structure, including, but not limited to nucleic acids, such as DNA and RNA, and peptides. The term refers to natural, synthetic and virtual molecules. The term includes compounds in a compound or a combinatorial library. The terms “ligand” and “molecule” are synonymous.

The terms “tissue-selective co-regulator” or “tissue-specific co-regulator” refer to a co-regulator that is expressed or otherwise present in a particular tissue preferentially or selectively over other tissues which may interact with the target molecule.

The terms “multiplicity of molecules,” “multiplicity of compounds,” or “multiplicity of containers” refer to at least two molecules, compounds, or containers.

The term “function” refers to the biological function of a target molecule, such as, e.g., a protein, peptide or polypeptide.

A “thermal unfolding curve” is a plot of the physical change associated with the unfolding of a protein or a nucleic acid as a function of temperature.

The terms “bind” and “binding” refer to an interaction between two or more molecules. More specifically, the terms refer to an interaction, such as noncovalent bonding, between a ligand and a target molecule, or a co-regulator and a target molecule, or a ligand, target molecule, and a co-regulator.

The term “modification of stability” refers to the change in the amount of pressure, the amount of heat, the concentration of detergent, or the concentration of denaturant that is required to cause a given degree of physical change in a target protein that is bound by one or more ligands, relative to the amount of pressure, the amount of heat, the concentration of detergent, or the concentration of denaturant that is required to cause the same degree of physical change in the target protein in the absence of any ligand. Modification of stability can be exhibited as an increase or a decrease in stability. Modification of the stability of a target molecule by a ligand indicates that the ligand binds to the target molecule.

The term “further modification of stability” refers to an additional modification of stability of the target molecule when in the presence of a molecule known to modify the stability of the target molecule and one or more additional molecules. More specifically, the one or more additional molecules can be co-regulators.

The term “unfolding” refers to the loss of structure, such as crystalline ordering of amino acid side-chains, secondary, tertiary, or quaternary protein structure. A target molecule, such as a protein, can be caused to unfold by treatment with a denaturing agent (such as urea, guanidinium hydrochloride, or guanidinium thiosuccicinate), a detergent, by treating the target molecule with pressure, by heating the target molecule, or by any other suitable change.

The term “physical change” encompasses the release of energy in the form of light or heat, the absorption of energy in the form or light or heat, changes in turbidity and changes in the polar properties of light Specifically, the term refers to fluorescent emission, fluorescent energy transfer, absorption of ultraviolet or visible light, change measurable by infrared spectroscopy or other spectroscopy methods, changes in the polarization properties of light, changes in the polarization properties of fluorescent emission, changes in the rate of change of fluorescence over time (i.e., fluorescence lifetime), changes in fluorescence anisotropy, changes in fluorescence resonance energy transfer, changes in turbidity, and changes in enzyme activity. Preferably, the term refers to fluorescence, and more preferably to fluorescence emission. Fluorescence emission can be intrinsic to a protein or can be due to a fluorescence reporter molecule. The use of fluorescence techniques to monitor protein unfolding is well known to those of ordinary skill in the art. For example, see Effink, M. R., Biophysical J. 66: 482-501 (1994).

An “unfolding curve” is a plot of the physical change associated with the unfolding of a protein as a function of parameters such as temperature, denaturant concentration, and pressure.

The term “modification of thermal stability” refers to the change in the amount of thermal energy that is required to cause a given degree of physical change in a target protein that is bound by one or more ligands, relative to the amount of thermal energy that is required to cause the same degree of physical change in the target protein in the absence of any ligand. Modification of thermal stability can be exhibited as an increase or a decrease in thermal stability. Modification of the thermal stability of a target molecule by a ligand indicates that the ligand binds to the target molecule.

The term “shift in the thermal unfolding curve” refers to a shift in the thermal unfolding curve for a target molecule that is bound to a ligand, relative to the thermal unfolding curve of the target molecule in the absence of the ligand.

The term “further shift in the thermal unfolding curve” refers to an additional shift of the thermal unfolding curve of the target molecule when in the presence of a molecule known to shift the thermal unfolding curve of the target molecule and one or more additional molecules. More specifically, the one or more additional molecules can be co-regulators.

The term “contacting a target molecule” refers broadly to placing the target molecule in solution with the molecule to be screened for binding. Less broadly, contacting refers to the turning, swirling, shaking or vibrating of a solution of the target molecule and the molecule to be screened for binding. More specifically, contacting refers to the mixing of the target molecule with the molecule to be tested for binding. Mixing can be accomplished, for example, by repeated uptake and discharge through a pipette tip. Preferably, contacting refers to the equilibration of binding between the target protein and the molecule to be tested for binding. Contacting can occur in the container or before the target molecule and the molecule to be screened are placed in the container.

The term “container” refers to any vessel or chamber in which the receptor and molecule to be tested for binding can be placed. The term “container” encompasses reaction tubes (e.g., test tubes, microtubes, vials, cuvettes, etc.). In embodiments of the invention, the term “container” refers to a well in a multiwell microplate or microtiter plate.

In embodiments of the invention, ligands that bind to the target molecule can be screened for their ability to bind to a target molecule in the presence of one or more tissue-selective co-regulators. The term “screening” refers generally to the testing of molecules or compounds for their ability to bind to a target molecule which is capable of denaturing or unfolding. The screening process can be a repetitive, or iterative, process, in which molecules are tested for binding to a protein in an unfolding assay.

As mentioned above, in accordance with embodiments of the invention, the tissue selectivity of a ligand for a co-regulator dependent target molecule can be identified based upon modification of stability of the target molecule. Ligands that modify the stability of the target molecule can be screened for their ability to further modify the stability of the target molecule in the presence of one or more tissue-selective co-regulators.

In an embodiment, to perform the screening, one or ligands (e.g. of a set) that modify the stability of the target molecule can be contacted with the target molecule and one of more tissue-selective co-regulators in each of a multiplicity of containers. The target molecule in each of the containers can then be treated to cause the target molecule to unfold. A physical change associated with the unfolding of the target molecule can be measured. An unfolding curve for the target molecule for each of containers can then be generated. Each of the unfolding curves may be compared to (1) each of the other unfolding curves and/or to (2) the unfolding curve for the target molecule in the absence of (i) any of the molecules from the set and/or (ii) the co-regulators.

Based upon the generated data, one can determine whether the screened ligands further modify the stability of the target molecule in the presence of the tissue-selective co-regulators, indicating whether a ligand is an agonist or an antagonist of the target molecule when the presence of a tissue-selective co-regulator. A further modification of stability of the target molecule is indicated by a further change in the unfolding curve of the target molecule.

In other embodiments of the invention, the tissue selectivity of a ligand for a co-regulator dependent target molecule can be determined by an analysis of molecules that modify the thermal stability, and more particularly, shift the thermal unfolding curve of the target molecule. Ligands that shift the thermal unfolding curve of a target molecule can be screened for their ability to further shift the thermal unfolding curve of the target molecule in the presence of one or more co-regulators.

In an embodiment of the invention, the screening can be accomplished by contacting the target molecule with one or more of ligands (e.g. of a set) that shift the thermal unfolding curve of the target molecule with one or more tissue-selective co-regulators in each of a multiplicity of containers. The multiplicity of containers can be heated, and a physical change associated with the thermal unfolding curve for the target molecule as a function of temperature can be measured for each of the containers. A thermal unfolding curve for the target molecule as a function of temperature can then be generated. The thermal unfolding curves that are generated can be compared with (1) each of the other thermal unfolding curves and/or to (2) the thermal unfolding curve for the target molecule in the absence of (i) any of the molecules from the set and/or (ii) the co-regulators.

In embodiments of the screening method, the containers can be heated in intervals, over a range of temperatures. The multiplicity of containers may be heated simultaneously. A physical change associated with the thermal unfolding of the target molecule can be measured after each heating interval. In an alternate embodiment of this method, the containers can be heated in a continuous fashion.

In embodiments of the invention, in generating an unfolding curve for the target molecule, a thermal unfolding curve can be plotted as a function of temperature for the target molecule in each of the containers.

In an embodiment of the invention, comparing the thermal unfolding curves can be accomplished by comparing the midpoint temperatures, Tₘ of each unfolding curve. The “midpoint temperature, Tₘ” is the temperature midpoint of a thermal unfolding curve. The Tₘ can be readily determined using methods well known to those skilled in the art. See, for example, Weber, P. C. et al., J. Am. Chem. Soc. 116:2717-2724 (1994); and Clegg, R. M. et al., Proc. Natl. Acad. Sci. U.S.A. 90:2994-2998 (1993).

For example, the Tₘ of each thermal unfolding curve can be identified and compared to the Tₘ obtained for (1) the other thermal unfolding curves and/or to (2) the thermal unfolding curve for the target molecule in the absence of (i) any of the molecules from the set and/or (ii) the co-regulators in the containers.

Alternatively or additionally, an entire thermal unfolding curve can be similarly compared to other entire thermal unfolding curves using computer analytical tools. For example, each entire thermal unfolding curve can be compared to (1) the other thermal unfolding curves and/or to (2) the thermal unfolding curve for the target molecule in the absence of (i) any of the molecules from the set and/or (ii) the co-regulators in the containers.

Based upon the generated data, one can determine whether any of the screened molecules further shift the thermal unfolding curve of the target molecule in the presence of a co-regulator, identifying whether a molecule is an agonist or antagonist of the target molecule when in the presence of a tissue-selective co-regulator. In this way, the tissue selectivity of the ligand for the target molecule can be determined.

The methods of the present invention that involve determining whether ligands that shift and/or further shift the thermal unfolding curve of a target molecule are distinct from methods that do not involve determining whether molecules shift and/or further shift the thermal unfolding curve of a target molecule, such as assays of susceptibility to proteolysis, surface binding by protein, antibody binding by protein, molecular chaperone binding of protein, differential binding to immobilized ligand, and protein aggregation. Such assays are well-known to those of ordinary skill in the art. For example, see U.S. Pat. No. 5,585,277; and U.S. Pat. No. 5,679,582. These approaches disclosed in U.S. Pat. Nos. 5,585,277 and 5,679,582 involve comparing the extent of folding and/or unfolding of the protein in the presence and in the absence of a molecule being tested for binding. These approaches do not involve a determination of whether any of the ligands that bind to the target molecule shift the thermal unfolding curve of the target molecule.

As discussed above, ligands that modify the stability of the target molecule can be screened for the ability to further modify the stability of the target molecule in the presence of a tissue-selective co-regulator. For example, ligands that are known to modify the stability of the target molecule can be screened against a panel of identified tissue-selective co-regulators for the target molecule, including co-activators and/or co-repressors. For convenience, the ligands known to modify the stability of the target molecule are referred to as a “set” of molecules.

If the stability of the target molecule is further modified in the presence of a ligand from the set and a tissue-selective co-activator of the target molecule as compared to the target molecule and the ligand from the set alone, then this is an indication that the ligand from the set is an agonist of the target molecule when in the presence of the tissue-selective co-activator. In this way, it can be determined that the ligand can act in agonist fashion for the target molecule in tissues that express the co-activator.

If the stability of the target molecule is further modified in the presence of a ligand from the set and a tissue-selective co-repressor of the target molecule as compared to the target molecule and the ligand from the set alone, then this is an indication that the ligand from the set is an antagonist of the target molecule when in the presence of the tissue-selective co-repressor. In this way, it can be determined that the ligand can act in antagonist fashion for the target molecule in tissues that express the co-repressor.

Similarly, ligands that shift the thermal unfolding curve of the target molecule can be screened for the ability to further shift the thermal unfolding curve of the target molecule in the presence of a tissue-selective co-regulator. For example, ligands that are known to shift the thermal unfolding curve of the target molecule can be screened against a panel of identified tissue-specific co-regulators for the target molecule, including co-activators and/or co-repressors. For convenience, the ligands that are known to shift the thermal unfolding curve of the target molecule are referred to as a “set” of molecules.

If the thermal unfolding curve of the target molecule is further shifted in the presence of a ligand from the set and a tissue-selective co-activator of the target molecule as compared to the target molecule and the ligand from the set alone, then this is an indication that the ligand from the set is an agonist of the target molecule when in the presence of the tissue-selective co-activator. In this way, it can be determined that the ligand can act in agonist fashion for the target molecule in tissues that express the co-activator.

If the thermal unfolding curve of the target molecule is further shifted in the presence of a ligand from the set and a tissue-selective co-repressor of the target molecule as compared to the target molecule and the ligand from the set alone, then this is an indication that the ligand from the set is an antagonist of the target molecule when in the presence of the tissue-selective co-repressor. In this way, it can be determined that the ligand can act in antagonist fashion for the target molecule in tissues that express the co-repressor.

The present invention also provides methods for determining the tissue selectivity of a ligand for a co-regulator dependent target molecule based on the lack of further modification of stability and/or a lack of further shift in the unfolding curve of a target molecule.

By “lack of further modification of stability of the target molecule” or “no further modification of stability of the target molecule,” it is meant that there is either an insignificant further change or no further change in the stability of the target molecule in the presence of both a ligand from the set and a co-regulator (as compared to the target molecule and the ligand from the set).

By “lack of further shift in the thermal unfolding curve of the target molecule” or “no further shift in the thermal unfolding curve of the target molecule,” it is meant that there is either an insignificant further change or no further change in the shift of the thermal unfolding curve of the target molecule in the presence of a ligand from the set and of a co-regulator (as compared to the target molecule and the ligand from the set).

In embodiments of the invention, whether a ligand acts in an antagonist fashion for a co-regulator dependent target molecule in a tissue can be identified based on the lack of further modification of stability and/or lack of further shift in the thermal unfolding curve of a target molecule when in the presence of a tissue-selective co-activator. In other embodiments of the invention, whether a ligand acts in an agonist fashion for a co-regulator dependent target molecule in a tissue can be identified based on the lack of further modification of stability and/or lack of further shift in the thermal unfolding curve of a target molecule when in the presence of a tissue-selective co-repressor.

A ligand can be identified as acting in antagonist fashion for a co-regulator dependent target molecule in a tissue by screening one or more of a set of ligands that modify the stability of the target molecule for their ability to further modify the stability of the target molecule in the presence of one or more tissue-selective co-activators. Methods for screening the ligands from the set for their effect on further modifying the stability of the target molecule are described above. If there is no further modification of the stability of the target molecule in the presence of a ligand of the set and a tissue-selective co-activator, then this is an indication that such ligand of the set can act in antagonist fashion for the target molecule in tissues that express the co-activator.

An antagonist can also be identified by screening one or more of a set of ligands that shift the thermal unfolding curve of the target molecule for their ability to further shift the thermal unfolding curve of the target molecule in the presence of one or more co-activators. Methods for screening one or more ligands of the set for their ability to further shift the thermal unfolding curve of the the target molecule are described above. If there is no further shift in the thermal unfolding curve of the target molecule in the presence of a ligand of the set and a tissue-selective co-activator, then this is an indication that such ligand of the set can act in antagonist fashion for the target molecule in tissues that express the co-activator.

A ligand can be identified as acting in agonist fashion for a co-regulator dependent target molecule in a tissue by screening one or more of a set of ligands that modify the stability of the target molecule for their ability to further modify the stability of the target molecule in the presence of one or more tissue-selective co-repressors. Methods for screening the ligands from the set for their effect on further modifying the stability of the target molecule are described above. If there is no further modification of the stability of the target molecule in the presence of a molecule of the set and a tissue-selective co-repressor, then this is an indication that such ligand of the set acts in agonist fashion for the target molecule in tissues that express the co-repressor.

A ligand can also be identified as acting in agonist fashion by screening one or more of a set of ligands that shift the thermal unfolding curve of the target molecule for their ability to further shift the thermal unfolding curve of the target molecule in the presence of one or more tissue-selective co-repressors. Methods for screening one or more ligands of the set for their ability to further shift the thermal unfolding curve of the the target molecule are described above. If there is no further shift in the thermal unfolding curve of the target molecule in the presence of a ligand of the set and a co-repressor, then this is an indication that such ligand of the set can act in agonist fashion for the target molecule in tissues that express the co-repressor.

The ability to use the present invention to determine the tissue selectivity of a ligand for a co-regulator dependent target molecule is based upon the ability of the present invention to identify the ligand as an agonist or an antagonist of the target molecule when in the presence of tissue-selective co-regulators.

Illustratively, one particular tissue, e.g. Tissue 1, may express a particular co-regulator, e.g. Co-regulator 1. A second particular tissue, e.g. Tissue 2, does not express Co-regulator 1 or expresses it at a different, i.e., lower level. If a ligand is determined by the methods of the present invention to be an agonist of the target molecule in the presence of Co-regulator 1, then the ligand can be expected to agonize the target molecule in Tissue 1 but not in Tissue 2, because Co-regulator 1 is active in Tissue 1 but substantially not in Tissue 2.

In another illustration, one particular tissue, e.g. Tissue 3, may express a particular co-regulator, e.g. Co-regulator 3. Another tissue, e.g. Tissue 4, expresses Co-regulator 3 and another co-regulator, e.g. Co-regulator 4. If a ligand is determined by the methods of the present invention to be an agonist of the target molecule in the presence of Co-regulator 3, but an antagonist of the target molecule in the presence of Co-regulator 4, it follows that the ligand can be expected to act as an agonist in Tissue 3 and a partial agonist in Tissue 4.

One can envision, by use of the methods of the present invention, various combinations or variations of these illustrations. For example, the methods of the present invention can be used to determine ligands that are agonists for some tissues but antagonists for other tissues, ligands that are partial agonists for some tissues but agonists for other tissues, ligands that are antagonists for some tissues but partial agonists for other tissues, etc.

The biological response of a ligand can be dependent upon the specific co-regulators that are present and their levels in a tissue-specific fashion. The designation of a ligand as an agonist, an antagonist, or a partial agonist is dependent upon the formation of an appropriate tertiary complex (ligand, target molecule, and co-regulator) and can be tissue-specific. The methods of the present invention can be used to identify the effect of ligands (e.g. identify agonists, antagonists, or partial agonists) on target molecules in a tissue-selective manner. The invention has particular utility in predicting the in vivo efficacy of drug lead ligands for particular tissues; i.e. tissue selective lead discovery for agonists and antagonists depending upon the tissue of interest.

Methods have been described above for the determination of tissue-selectivity of ligands for a co-regulator dependent target molecule based on providing ligands that are known to modify the stability and/or shift the thermal unfolding curve of the target molecule and screening such ligands for their ability to further modify the stability of and/or shift the thermal unfolding curve of the target molecule. The invention also encompasses methods for the providing of such ligands in conjunction with the identification of their tissue-selectivity. Such methods are particularly useful in identifying such ligands for orphan receptors, for which ligands that bind to the receptor are not known.

Ligands that modify the stability and/or shift the thermal unfolding curve of the target molecule (referred to above as a “set” for convenience) can be obtained by the screening of a multiplicity of different molecules. For example, ligands that modify the stability of the target molecule can be obtained by the screening of one or more of a multiplicity of different molecules for their ability to modify the stability of the target molecule. Similarly, molecules that shift the thermal unfolding curve of the target molecule can be obtained by the screening of one or more of a multiplicity of different molecules for their ability to shift the thermal unfolding curve of the target molecule. In embodiments of the invention, the number of molecules that can be screened range from about one thousand to one million.

Molecules can be screened for their ability to modify the stability of the target molecule by a method similar to the screening method described above for determining tissue selectivity of a ligand. For example, the target molecule can be contacted with one or more of a multiplicity of different molecules in each of a multiplicity of containers. The target molecule in each of the multiplicity of containers can be treated to cause it to unfold. A physical change associated with the unfolding of the target molecule can be measured. An unfolding curve for the target molecule for each of the containers can be generated. Each of these unfolding curves can be compared to (1) each of the other unfolding curves and/or to (2) the unfolding curve for the target molecule in the absence of any of the multiplicity of different molecules.

Based upon the generated data, one can determine whether any of the screened molecules modify the stability of the target molecule. A modification of stability of the target molecule is indicated by a change in the unfolding curve of the target molecule. If a molecule modifies the stability of the target molecule, it can then be screened to determine its tissue-selectivity for the target molecule by the methods described above.

Similarly, molecules can be screened for their ability to shift the thermal unfolding curve of the target molecule by a method similar to the screening method for determining tissue selectivity. For example, the target molecule can be contacted with one or more of a multiplicity of different molecules in each of a multiplicity of containers. The containers can be heated, and a physical change associated with the thermal unfolding of the target molecule can be measured in each of the containers. A thermal unfolding curve for the target molecule can be generated as a function of temperature for each of the containers.

The thermal unfolding curves can be compared with (1) each of the other thermal unfolding curves and/or to (2) the thermal unfolding curves for the target molecule in the absence of any of the multiplicity of different molecules. In embodiments of the invention, the Tₘ of each thermal unfolding curve can be identified and compared to the Tₘ obtained for (1) the other thermal unfolding curves and/or to (2) the thermal unfolding curve for the target molecule in the absence of any of the multiplicity of molecules. Alternatively, each entire thermal unfolding curve can be compared to (1) the other thermal unfolding curves and/or to (2) the thermal unfolding curve for the target molecule in the absence of any of the multiplicity of different molecules.

Based upon the generated data, one can determine whether any of the screened molecules shift the thermal unfolding curve of the target molecule. If a molecule shifts the thermal unfolding curve of the target molecule, it can then be screened to determine its tissue selectivity for the target molecule by the methods described above.

As mentioned above, the methods of the present invention are particularly useful in identifying ligands for orphan receptors, for which ligands that bind to the receptor are not known. Similarly, the invention provides for a methods for identifying agonists and antagonists of a target molecule having an unknown function in a tissue-selective manner.

In an embodiment of the invention, a set of ligands is provided that modify the stability of a target molecule having an unknown function. This set of ligands modifies the stability of receptors which share biological function. The set of ligands that modify the stability of the target molecule can be provided by screening one or more panels of molecules which modify the stability of receptors which share biological function for their ability to modify the stability of the target molecule. Methods for providing such a set of ligands are described in more detail in U.S. Patent Publication No. US 2001/0003648, herein incorporated by reference in its entirety.

One or more ligands of the set can be screened for their ability to further modify the stability of the target molecule in the presence of one or more tissue-selective co-regulators. As discussed in detail above, a further modification of the stability of the target molecule in the presence of a molecule of the set and a tissue-selective co-regulator indicates whether the molecule acts in agonist or antagonist fashion for the target molecule in a tissue-selective manner. Embodiments of the invention also include an identification of agonist and antagonist ligands in a tissue selective manner based upon no further modification of stability of the target molecule.

In another embodiment of the invention, a set of ligands are provided that shift the thermal unfolding curve of a target molecule having an unknown function. This set of ligands shifts the thermal unfolding curve of receptors which share biological function. The set of ligands that shift the thermal unfolding curve of the target molecule can be provided by screening one or more panels of molecules which shift the thermal unfolding curve of receptors which share biological function for their ability to shift the thermal unfolding curve of the target molecule. Methods for providing such a set of molecules are also described in more detail in U.S. Patent Publication No. US 2001/0003648.

One or more molecules of the set can be screened for their ability to further shift the thermal unfolding curve of the target molecule in the presence of one or more co-regulators. As discussed in detail above, a further shift in the thermal unfolding curve of the target molecule in the presence of a molecule of the set and a tissue-selective co-regulator indicates whether molecule acts in agonist or antagonist fashion for the target molecule in a tissue-selective manner. Embodiments of the invention also include an identification of agonist and antagonist ligands in a tissue selective manner based upon no further shift in the thermal unfolding curve of the target molecule.

In embodiments of the invention, a microplate thermal shift assay is a particularly useful means for identifying ligands and identifying such ligands as tissue-selective agonists or antagonists of co-regulator dependent target molecules. The microplate thermal shift assay is a direct and quantitative technology for assaying the effect of one or more molecules on the thermal stability of a target receptor.

The theory, concepts, and application of the microplate thermal shift assay, and apparatuses useful for practicing the microplate thermal shift assay are described in U.S. Pat. Nos. 6,020,141; 6,036,920; 6,291,191; 6,268,218; 6,232,085; 6,268,158; 6,214,293; 6,291,192; and 6,303,322, which are all hereby incorporated by reference in their entireties. The microplate thermal shift assay discussed in these references can be used to implement the screening methods described above.

The microplate thermal shift assay provides a thermodynamic readout of ligand binding affinity. The assay depends upon the fact that each functionally active target molecule is a highly organized structure that melts cooperatively at a temperature that is characteristic for each target molecule and representative of its stabilization energy. When a molecule binds to a target molecule, the target molecule is stabilized by an amount proportional to the ligand binding affinity, thus shifting the midpoint temperature to a higher temperature.

There are many advantages to using the thermal shift assay since it does not require radioactively labeled compounds, nor fluorescent or other chromophobic labels to assist in monitoring binding. The assay takes advantage of thermal unfolding of biomolecules, a general physical chemical process intrinsic to many, if not all, drug target biomolecules. General applicability is an important aspect of this assay, as it obviates the necessity to invent a new assay every time a new therapeutic receptor protein becomes available.

Further, using the thermal shift assay, owing to the proportionality of the Tₘ and the ligand binding affinity, ligand binding affinities ranging from greater than 10 micromolar to less than 1 nanomolar can be measured in a single well experiment. Thus, the thermal shift assay can be used to quantitatively detect ligand binding affinity to a target molecule alone and/or in the presence of a co-regulator.

Further, the thermal shift assay can be used in the identification of tissue-selective agonists and antagonists (as well as partial agonists) on a quantitative basis based upon the change in the Tₘ between the ligand and target molecule and the ligand, target molecule and a co-regulator. The microplate thermal shift assay can be used to measure multiple ligand binding events on a single target molecule as incremental or additive increases of the target molecule's melting temperature.

The present invention has particular utility in the identification of ligands and the identification of such ligands as agonist or antagonist in nuclear receptors in a tissue-selective manner. For example, the present invention may be used to determine binding affinities for nuclear receptor ligands to predict in vivo efficacy, to discriminate ligands as agonist or antagonist to predict biological response, to identify ligands for orphan receptors to discover their biological function, and to determine tissue specificity by analyzing the preferential recruitment of co-regulators.

For example, the present invention may be used to identify ligands that interact with the ligand binding domain of ER-α and ER-β, the two subtypes of the estrogen receptor family. These domains contain two known binding sites, one for estrogen like compounds and another for co-regulator proteins. The present invention can be used to identify ligands that interact with the estrogen receptor. These ligands produce an observed increase in the stability of the receptor which is proportional to the inherent affinity of the ligand.

The ligand binding domain of nuclear receptors, and co-regulator proteins can be expressed using standard recombinant methods in Escherichia coli. Co-regulator peptides can be synthesized using standard methods. The melting temperature of the purified protein of interest can be determined by the microplate thermal shift assay in the absence and in the presence of small molecule ligands.

Molecules are provided that stabilize the target molecule of interest. Such small molecules can be obtained by screening in the microplate thermal shift assay, as referred to above. The number of small molecules in the screen can range from about one thousand to one million. The small molecules can be natural or synthetic.

Once a set of small molecules have been identified to stabilize the protein of interest, then these molecules can be screened against a panel of co-regulators, such as proteins or peptide fragments, to measure their effect on the thermal stability of the protein. If a synergistic effect is observed, the compounds can be classified as agonist or antagonist Equilibrium constants are calculated for both ligand and co-regulator and related to biological responses.

For assigning biological function to orphan receptors, the rate limiting step is the generation of a tool compound. One can screen the receptor of interest against a panel of compounds and identify ligands that stabilize the receptor of interest by the methods described above. Once ligands are identified, then one can screen against co-regulators to determine if the identified ligand is an agonist or an antagonist the methods described above, and identify preferred co-regulators that produce a maximal response.

Cell lines that contain the receptor of interest, as determined by, e.g. Western blot analysis, can be treated with the identified ligand. The ligand treated cell line can then be profiled for gene expression with DNA chips and compared against untreated cell lines. If the identified ligand is an agonist, a number of genes would be expected to be up-regulated when compared against the untreated cell line. If the identified ligand is an antagonist, a number of genes would be expected to be down-regulated when compared against the untreated cell line. Once this information is generated, the biological function of the receptor can be defined This information, with the combination of chemi-informatics and bio-informatics can also assist in developing therapeutic hypothesis and testing them for the treatment of disease (see, e.g., Giguere, Endocrine Reviews 20:689-725 (1999), incorporated by reference herein in its entirety.)

The present invention also encompasses the use of the screening methods described above for determining gene specificity. By “gene-specific,” “gene specificity,” “gene-selective,” or “gene selectivity,” it is meant that one can target the expression or repression of a particular gene based upon the recruitment of a specific co-regulator which interacts with the target molecule (such as, e.g. a nuclear receptor) and activates or represses transcription of the particular gene.

For example, methods for identifying an agonist or an antagonist of a co-regulator dependent target molecule based upon modification of stability and/or shift in the thermal unfolding curve of the target molecule have been described in detail above. Illustratively, one may determine using the present invention whether a ligand is an agonist or an antagonist of a target molecule when in the presence of a particular co-regulator by providing a set of molecules that modify the stability of and/or shift the thermal unfolding curve of the target molecule and screening one or more molecules of the set for their ability to further modify the stability and/or further shift the thermal unfolding curve of the target molecule in the presence of a particular co-regulator.

Based on the identification of the ligand as an agonist or an antagonist of the target molecule when in the presence of particular co-regulators, one can determine the gene specificity of a ligand for a target molecule. For example, a particular gene, e.g. “Gene A,” may be produced when the target molecule interacts with a particular co-activator present or expressed by a tissue, e.g. “Co-activator A.” A second gene, e.g “Gene B,” may be produced when the target molecule interacts with a second co-activator present or expressed in the tissue, e.g “Co-activator B.”

If one wants to stimulate the production of one of Gene A or Gene B without substantially stimulating the other, one can use the present invention to determine whether a ligand further modifies the stability and/or further shifts the thermal unfolding curve of the target molecule in the presence of one of the co-activators (and thus identifying the ligand as an agonist for that co-activator) and substantially not the other. In this way, one can determine whether a ligand can selectively effect the production of a specific gene.

Although the ligand binding domain of nuclear receptors, ligands and co-regulators that interact with this domain is described, the invention can be extended to the full length protein, in the presence of additional regulators and finally in the presence of DNA.

Further, it must be emphasized that the methods and the thermodynamic principles for data analysis can be used for any protein-protein interaction whose affinity is modulated by ligands or allosteric regulators. Examples can be and are not limited to GPCR's interacting with G-proteins to discriminate agonist from antagonist ligands; discriminating compounds that antagonize the association of SH2 domains to phophorylated forms of protein tyrosine kinases; identifying compounds that agonize or antagonize the PKA holoenzyme by affecting the oligomeric state of the enzyme; discriminating compounds that promote or inhibit the association of NF-κB to IκB; or compounds that promote or inhibit the oligomerization of transcription factors.

Also, these studies are not limited for protein-protein interactions but also can be used for protein-peptide interactions where the peptides represent short linear sequences representing protein domains that interact preferentially with the protein of interest.

Further Discussion of Tissue Specificity and Partial Agonism

Gene activation or repression can be restricted in a single tissue. Therefore, discrimination within the family of co-activators and co-regulators is required for a gene specific response. Such is an example of the transcription of the uncoupling protein-1 (UCP1) that is present only in brown adipose tissue and requires the nuclear receptor PPAR-γ and the co-activator PGC-1 (Oberkofier H., et al., J. Biol. Chem. 277:16750-16757 (2002)).

However, not all genes activated in brown adipose tissue by PPAR-γ are mediated through PGC-1 (Puigserver, P., et al., Cell 92:829-839 (1998); Wu et al., Cell 98:115-124 (1999); Rosen E. D., et al., Genes & Devel., 14:1293-1307 (2000)). There is a set of nuclear receptor ligands that partially activate or repress a set of genes and they are referred as partial agonist, such as ligands that activate the members of the peroxisome, proliferator activated nuclear receptor family (PPAR's) (Camp, H. S., et al., Diabetes 49:539-547 (2000)).

The molecular basis of partial agonism is not clearly understood but it can interpreted with one of three mechanisms: i) the ligand induces a conformational change of the receptor with reduced affinity for co-activator ii) the absence of a specific co-activator in a given tissue resulting in a reduced biological response or iii) the relative expression levels of co-activators and co-repressors competing for ligand occupied or ligand free nuclear receptor, Therefore the biological response induced by ligands on nuclear receptors can be regulated on the context of tissue specificity for a given co-regulator and also on the relative levels of a given co-activator and co-repressor protein present in a given tissue.

### Further Embodiment of the Invention

Receptors, such as nuclear receptors, can exert biological function in the absence of a ligand. The function may be a repression or an activation of a function, depending on their ability to interact with co-regulators.

A receptor that activates gene expression in the absence of a ligand will interact with appreciable affinity with a co-activator protein Such a receptor may be referred to as constitutively active. In contrast, a receptor that represses gene expression in the absence of a ligand will interact with appreciable affinity with a co-repressor protein. Such a receptor is referred to as a repressor.

By use of the methods of the present invention, one can screen the receptor in the absence of a ligand against a panel of co-regulators to determine the natural state of the receptor in a tissue specific fashion.

For example, using the methods of the present invention, one can screen a panel of co-activators and/or co-repressors for their ability to modify the stability of and/or shift the thermal unfolding curve of the receptor in the absence of a ligand by the methods described above. If the stability of the receptor is modified or the thermal unfolding curve of the receptor is shifted when in the presence of a co-activator, it may be concluded that the receptor is constitutively active when in presence of the co-activator. If the stability of the receptor is modified or the thermal unfolding curve of the receptor is shifted when in the presence of a co-repressor, it may be concluded that the receptor is a repressor in its unliganded state when in the presence of the co-repressor.

The ability to use the present invention to determine the natural state of the receptor in a tissue-selective fashion is based upon the ability of the present invention to identify whether the stability or thermal unfolding curve of the receptor is affected when in the presence of tissue-selective co-regulators.

Illustratively, one particular tissue, e.g. Tissue 1, may express a particular co-regulator, e.g. Co-regulator 1. A second particular tissue, e.g. Tissue 2, does not express Co-regulator 1 or expresses it at a different, lower level. If the stability of a receptor is modified or the thermal unfolding curve of the receptor is shifted in the presence of Co-regulator 1, then the receptor can be expected to be constitutively active in Tissue 1 but not in Tissue 2, because Co-regulator 1 is active in Tissue 1 but substantially not in Tissue 2.

Having now generally described the invention, the same will become more readily understood by reference to the following specific examples which are included herein for purposes of illustration only and are not intended to be limiting unless otherwise specified.

### EXAMPLES

Experimental Results For Nuclear Receptors

As discussed above, the biological response of nuclear receptors is mediated by DNA binding and recruitment of the appropriate ancillary transcription factors, such as co-regulators. See, e.g., Robyr D., et al., Mol. Endo. 14:329-347 (2000); Lee, J. W., et al, Cell. Mol. Life Sci. 58:289-297 (2001); and Rosenfeld, M. G. & Glass, C. K, J. Biol. Chem. 276:36865-36868 (2001). Co-regulators activate (co-activators) or repress (co-repressor) gene expression. Ligands, when bound to nuclear receptors induce conformational changes that can result in preferential recruitment of a co-regulator protein. In the case of an agonist ligand, co-activators are recruited, resulting in gene activation. In the case of an antagonist ligand co-repressors are recruited resulting in gene repression.

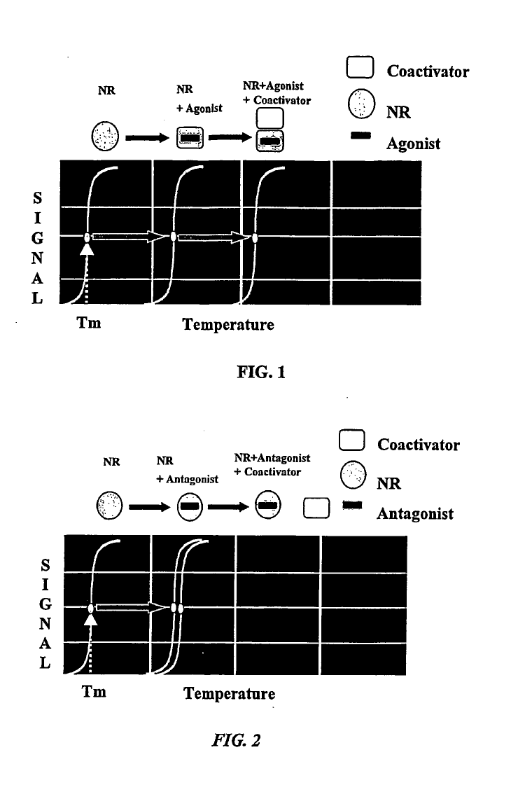

The experimental results expected for an agonist response vs. an antagonist response in the presence of a co-activator is shown in FIGS. 1 and 2. In the case of an agonist ligand and in the presence of co-activator protein/peptide the prediction is an increase in the stability of the receptor (FIG. 1), while for an antagonist no additional stabilization will be observed (FIG. 2).

### Example 1

Table 1, shown below, is a summary of the data obtained for ER-α and ER-β for the study of a panel of four known agonist and three known antagonists in the presence of a co-activator protein SRC-3; in the presence of two co-activator peptides SRC1-NR2 and SRC3-NR2 derived from the sequence of the co-activators SRC-1 and SRC-3; and in the presence of the co-repressor peptide NCoR-1 derived from the co-repressor NCoR-1.

The concentration of ER-α and ER-β in all of the experiments was 8 μM, the ligand concentration was 20 μM, SRC-3 was 11 μM, and the co-regulator peptides SRC1-NR2, SRC3-NR2, and NCoR-1 was at 100 μM. The experiments were performed in 25 mM HEPES buffer pH 7.9, 200 mM NaCl, 5 mM DTT and in the presence of 25 μM dapoxyl sulfonamide or ANS dye (available from Molecular Probes, Inc., Eugene, Oreg.).

A 2 μL ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure nixing of the protein-dye and ligand solutions followed by layering of 1 μL of silicone oil to prevent evaporation during heating of the samples. Data were collected on a Thermofluor apparatus (see, e.g., U.S. Pat. Nos. 6,020,141; 6,036,920; 6,291,191; 6,268,218; 6,232,085; 6,268,158; 6,214,293; 6,291,192; and 6,303,322) and analyzed using non-linear least squares fitting software. The results listed below are the average of four experiments. The values for the co-regulators represent a change in Tₘ stabilization from the receptor-ligand ΔTₘ values.

TABLE 1
Observed ΔTm Stabilization of Estrogen Receptors
in the presence of ligands and co-regulators

| | | | SRC1- | SRC3- | NcoR1- |
|---|---|---|---|---|---|
| | — | SRC-3 | NR2 | NR2 | NR1 |
| ER-α | 0.0 | 1.5 | 0.8 | 0.9 | 0.0 |
| Estradiol | 14.8 | 3.8 | 4.9 | 4.3 | 0.0 |
| Estrone | 7.7 | 3.5 | 3.0 | 2.3 | −0.3 |
| 17-α-ethylene-E2 | 15.5 | 4.5 | 4.5 | 3.9 | −0.1 |
| 2-methoxy-E2 | 3.5 | 5.3 | 5.5 | 4.3 | −0.7 |
| tamoxifen | 8.5 | 1.1 | −0.5 | 0.0 | 0.1 |
| 4-OH-tamoxifen | 17.7 | 0.2 | 0.2 | 0.7 | 0.1 |
| ICI-182780 | 13.9 | 0.5 | 0.2 | 0.2 | −0.6 |
| ER-β | 0.0 | 0.9 | 0.7 | 0.9 | −0.4 |
| Estradiol | 17.5 | 1.7 | 3.4 | 3.5 | 0.0 |
| Estrone | 11.3 | 1.6 | 3.8 | 3.7 | −0.3 |
| 17-α-ethylene-E2 | 15.3 | 2.6 | 2.6 | 2.6 | −0.7 |
| 2-methoxy-E2 | 2.9 | 2.5 | 4.4 | 4.4 | −0.7 |
| tamoxifen | 9.8 | 1.3 | 0.2 | 0.4 | 0.4 |
| 4-OH-tamoxifen | 18.2 | 1.2 | 0.2 | 0.8 | 0.3 |
| ICI-182780 | 16.7 | 0.9 | 0.4 | 0.6 | 0.3 |

From the above results, from counter-screening in the presence of co-activator protein/peptide in the presence of the estrogen-like compounds, an additional stabilization was observed for both receptors. Thus, these compounds act like agonists in agreement with literature. The tamoxifen and ICI compound are known antagonists and they have no ability to recruit co-activators. This is also in agreement with the literature.

Also, the co-activator SRC-3 is preferentially recruited by ER-α vs. ER-β. Therefore, the prediction is that these estrogen like compounds have a higher biological response in cell lines that contain ER-α vs. ER-β in the presence of SRC-3.

Further, the estrogen receptor does not have ability to recruit co-repressor peptide, therefore from a biological point of view the prediction is that gene repression will occur in ligand dependent fashion.

### Example 2

ER-α was screened against a panel of steroid-like ligands to verify the ability of the methods of the present invention to determine ligands, and the function (see, e.g., U.S. Patent Publication No. US 2001/0003648 A1), of ER-α if this receptor was classified as an orphan. Ligands that are known to interact with ER-α are identified as producing an increase in the stability of the receptor (compounds that are underlined versus those which are not underlined).

The concentration of ER-α in all of the experiments was 8 μM and the ligand concentration was 20 μM. The experiments were performed in 25 mM phosphate pH 8.0, 200 nM NaCl, 10% glycerol and in the presence of 25 μM dapoxyl sulfonamide dye (available from Molecular Probes, Inc., Eugene, Oreg.).

A 2 μL ligand solution at 2 times the final concentration was dispensed with a micropipette into a 384 well black wall Greiner plate. Then, 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus (see, e.g., U.S. Pat. Nos. 6,020,141; 6,036,920; 6,291,191; 6,268,218; 6,232,085; 6,268,158; 6,214,293; 6,291,192; and 6,303,322) and analyzed using non-linear least-squares fitting software. The results listed in Table 2, shown below, are the average of four experiments.

TABLE 2
Summary of data for ER-α in the presence of a panel of steroid ligands

| Steroid Ligand | ΔTm | Targeted Receptor |
|---|---|---|
| 4-androstene | −0.23 | androgen receptor |
| androsterone | 0.23 | androgen receptor |
| corticosterone | −0.27 | glucocorticoid receptor |
| cortisone | 0.01 | glucocorticoid receptor |
| β-estradiol | 15.19 | estrogen receptor |
| estrone | 9.91 | estrogen receptor |
| 17-α-ethylene-estradiol | 18.72 | estrogen receptor |
| 17-α-hydroxyprogesterone | −0.21 | progesterone receptor |
| 2-methoxy-estradiol | 5.98 | estrogen receptor |
| quabain | −0.21 | progesterone receptor |
| progesterone | −0.19 | progesterone receptor |
| 4-OH-tamoxifen | 19.99 | estrogen receptor |

If ER-α was an orphan receptor, the data would had been interpreted that this receptor is a member of the estrogen receptor family. If the identified ligands that bind to the receptor had been screened against a panel of co-regulators, as in Example 1, β-estradiol, estrone, 17-α-ethyleneestradiol, and 2-methoxyestradiol are agonists for this receptor, while 4-hydroxytamoxifen is an antagonist This data set demonstrates the utility of the microplate thermal shift assay for the identification of ligands for orphan receptors.

### Example 3

Examples of other protein-protein interactions that may be analyzed using the present invention are illustrated in Table 3, shown below.

TABLE 3

| | Protein Partner | Ligand | Related Biological |
|---|---|---|---|
| Protein of Interest | (co-regulator) | Phenotype | Activity |
| GPCR | Gsα | Agonist | Increase cAMP or |
| | | | stimulate regulation of |
| | | | Ca²⁺ channels |
| GPCR | Giα | Agonist | Decrease cAMP |
| GPCR | Goα | Agonist | Stimulate regulation of |
| | | | Ca²⁺ channels |
| GPCR | Gta | Agonist | Increase cGMP and |
| | | | phosphodiesterase |
| | | | activity |
| GPCR | Gqα | Agonist | Increase phospholipase |
| | | | Cβ activity |
| GPCR | Gsα | Antagonist | No effect on basal |
| | | | activity, or decrease |
| | | | cAMP, or inhibition of |
| | | | Ca²⁺ channel |
| | | | stimulation |
| GPCR | Giα | Antagonist | No effect on basal |
| | | | activity, or increase |
| | | | cAMP |
| GPCR | Goα | Antagonist | No effect on basal |
| | | | activity, or inhibition of |
| | | | Ca²⁺ channel |
| | | | stimulation |
| GPCR | Gtα | Antagonist | No effect on basal |
| | | | activity, or decrease |
| | | | cGMP and |
| | | | phosphodiesterase |
| | | | activity |
| GPCR | Gqα | Antagonist | No effect on basal |
| | | | activity, or decrease |
| | | | phospholipase Cβ |
| | | | activity |
| Src | SH2 | Antagonist | Inhibition of osteoclast |
| | | | mediated resoprtion of |
| | | | bone |
| Src | SH2 | Agonist | Stimulation of |
| | | | osteoclast mediated |
| | | | resorption of bone |
| Jac | SOCS | Agonist | Gene transcription |
| Jac | SOCS | Antagonist | Gene repression |
| NF-κB | IκB | Antagonist | Gene transcription |
| NF-Kb | IκB | Agonist | Gene repression |

Different embodiments of this invention can include and are not limited to the examples above. The general nature of the examples contain the protein of interest, the interacting protein or peptide partner (co-regulator, e.g., a co-activator or co-repressor), and the ligand that can enhance (an agonist) or inhibit (an antagonist) the interaction in a tissue-selective manner.

### Example 4

The steroid receptor coactivator family (SRC) consists of three members designated as SRC-1, SRC-2 and SRC-3. SRC-3 is expressed in a tissue specific fashion and is present mainly in mammary glands, oocytes, smooth muscle, hepatocytes and vaginal epithelium (Xu et al., Nat. Acad. Sci. USA 97:6379-6384 (2000)). On the other hand SRC-1 is highly expressed in cardiac muscle and the neocortex while SRC-3 is absent in those tissues (Misiti, S., et al., Endocrinology 140:1957-1960 (1999)); and SRC-3 is expressed in mammary cells while SRC-1 is not (Shang, Y. and Brown, M., Science 295:2465-2468 (2002)). Decreased organ growth in the four steroid responsive tissues was observed in deficient mice for SRC1 or SRC2, while in SRC3 knockout mice had defective hormonal signaling pathways. These data indicate that these co-activators do possess functional specificity.

Table 4, shown below, is a summary of the data obtained for ER-α and ER-β in the presence of the coactivator proteins SRC-1 SRC-2 and SRC-3 and in the presence of seven ligands. The concentration of ER-α in all experiments was 8 μM, the ligand concentration was 40 μM, SRC-1 and SRC-3 were at 20 μM. The experiments were performed in 25 mM HEPES pH 7.9, 200 mM NaCl, 5 mM DTT and in the presence of 25 μM dapoxyl sulfonamide or ANS dye (available from Molecular Probes, Inc., Eugene, Oreg.).

A 2 μL ligand solution at 2 times the final concentration was dispensed with a micropipette into a 384 well black wall Greiner plate. Then 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus and data were analyzed using software that employs a non-linear Marquardt algorithm. Reported results are the average of four experiments. The values for the co-regulators represent a change in Tₘ stabilization from the receptor-ligand ΔTₘ values.

TABLE 4
Observed ΔTₘ Stabilization of ER-α in the Presence
of Ligands and the Co-activator Proteins SRC-1 and SRC-3

| Ligand | | SRC1 | SRC2 | SRC3 |
|---|---|---|---|---|
| ER-α | | 1.1 | 1.0 | 1.5 |
| Agonist | | | | |
| Estradiol | 14.8 | 1.6 | 3.9 | 3.8 |
| Estrone | 7.7 | 1.4 | 2.2 | 3.5 |
| 17-α-ethylene- | 15.5 | 1.6 | 3.7 | 4.9 |
| estradiol | | | | |
| 2-methoxy- | 3.5 | 2.1 | 4.2 | 5.3 |
| estradiol | | | | |
| Antagonist | | | | |
| Tamoxifen | 8.5 | 1.1 | 0.7 | 1.1 |
| 4-OH-Tamoxifen | 17.1 | 0.2 | 0.2 | 0.2 |
| ICI-182780 | 13.9 | 0.4 | 0.5 | 0.5 |
| ER-β | | 0.7 | 0.9 | 0.9 |
| Agonist | | | | |
| Estradiol | 17.5 | 0.5 | 1.9 | 1.7 |
| Estrone | 11.3 | 0.7 | 1.5 | 1.5 |
| 17-α-ethylene- | 15.3 | 1.1 | 2.5 | 2.6 |
| estradiol | | | | |
| 2-methoxy- | 2.9 | 1.0 | 2.4 | 2.5 |
| estradiol | | | | |
| Antagonist | | | | |
| Tamoxifen | 9.8 | 1.1 | 1.1 | 1.3 |
| 4-OH-Tamoxifen | 18.2 | 0.9 | 1.0 | 1.2 |
| ICI-182780 | 16.7 | 0.5 | 0.6 | 0.9 |

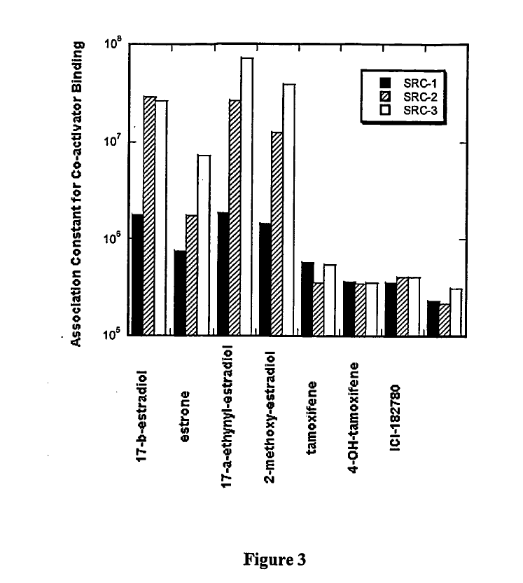

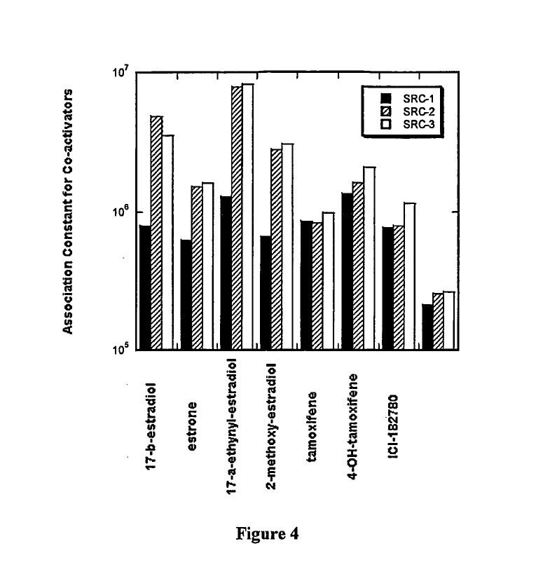

FIG. 3 illustrates binding constants, Ka, for co-activator proteins SRC-1, SRC-2 and SRC-3 in the presence of ER-α ligands. FIG. 4 illustrates binding constants, Ka, for co-activator proteins SRC-1, SRC-3 and SRC-3 in the presence of ER-β ligands Binding constants were calculated from the observed induced ligand and co-regulator stabilization of the nuclear receptor. Binding constants for SRC-3 in the presence of agonist are on average 5 to 20 times higher than for SRC-1. The observed binding constants for SRC-1 in the presence of agonist are equal to or two-fold higher than those for the co-activators in the presence of the antagonist.

From Table 4 and FIGS. 3 and 4 we can conclude the following:

a) Counterscreening in the presence of the co-activator proteins SRC-1, SRC-2 and SRC-3 and in the presence of the estrogen like compounds we observe and additional stabilization for both receptors. The conclusion based on the experimental results is that these compounds act like agonist for ER-α and ER-β with the exception of the ER-β and SRC-1 interactions.

b) Tamoxifen, 4-OH-tamoxifen and ICI-182,780 are known antagonists and have very little ability to recruit co-activator (this is in agreement with literature).

c) The preferential recruitment for the co-activators for ER-α is in the order of SRC-3>SRC-2>SRC-1, with the exception of the estradiol ligand the SRC-3 and SRC-2 interactions are equally potent.

d) The preferential recruitment for the co-activators for ER-β is in the order of SRC-3=SRC-2>SRC-1.

e) Agonists recruit SRC-1 equally as well as the antagonists for SRC-1 and SRC-3 for both ER-α and ER-β.

f) The estradiol ligands are more potent as agonists in the presence of SRC-3 and SRC-2 than SRC-1 in the context of the ternary complexes ER-α: agonist:SRC-3 vs. ER-α:antagonist:SRC-1; ER-β:agonist:SRC-3 vs. ER-β:antagonist:SRC-1; ER-α:agonist:SRC-2 vs. ER-α:antagonist:SRC-1; ER-β:agonist:SRC-2 vs. ER-β:antagonist:SRC-1.

g) SRC-3 and SRC-2 are preferentially recruited by ER-α agonist complexes than ER-β agonist complexes.

h) On average, ER-α agonist recruit the SRC family of co-activators 20-100 fold higher than the receptor in the absence or presence of an antagonist ligand, while for ER-β the enhancement in affinity is only five-fold.

ER-α agonist ligands favor the recruitment of SRC-3 vs. SRC-1. The prediction based on this observation is that these agonists will be more efficient in activating genes in tissues where SRC-3 is present. Since tamoxifen and 4-OH-tamoxifen are known antagonists for ER-α and they recruit SRC-3 and SRC-1 as efficiently the agonists do for SRC-1 the prediction is that these agonist ligands have no biological response in tissues that express SRC-1 and not SRC-3.

ER-α in the presence of the agonist estrone binds SRC-3 less tightly than the other agonist ligands do. The prediction is that this ligand might be a partial agonist for ER-α in tissues where SRC-3 is present when compared to the other agonist ligands. The differences in affinities for co-activators for the agonist occupied and the ligand free receptors, implies the biological activity of ER-α is more tightly regulated by endogenous concentration of ligands while for ER-β is mostly un-affected. Therefore, the differential biological response of an agonist ligand will be highly dependent on the formation of the appropriate ternary complex and the affinity of the ligand occupied receptor for co-regulators, and the relevant concentrations of proteins and endogeneous ligands.

### Example 5

A partial agonist is a ligand that produces a sub-maximal response even at full receptor occupancy. It also antagonizes a full agonist down to levels of its own stimulated biological response. The molecular basis for this is not known, but it is believed that it can be dependent on the relative expression levels of co-activator and co-repressors and the relative affinities for their co-regulators.

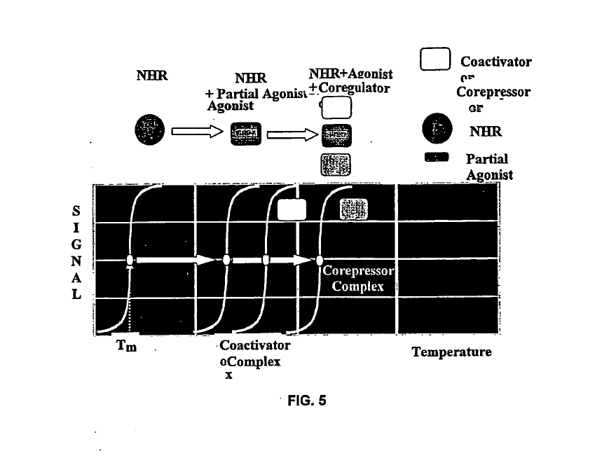

Based on the experimental hypothesis of the scheme illustrated in FIG. 5, a partial agonist can induce a conformational change to the nuclear receptor to recruit co-activator or co-repressors with different affinities. The ratio of these affinities will dictate if a biological response will be observed.

For instance, if a ligand strengthens the interaction for co-activator and weakens the interaction for co-repressor then it will have a biological response of a partial agonist If a ligand strengthens the interaction for co-activator and abolishes binding for co-repressor then one will have an agonist If a ligand affects equally binding for co-activator and co-repressor then there will be no biological response.

Table 5, shown below, is a summary of the data obtained PPAR-γ in the presence of the co-activator peptide SRC1-NR2 and the co-repressor peptide NCoR-1, and in the presence of ligands.

The concentration of PPAR-γ in all experiments was 8 μM, the ligand concentration was 40 μM, SRC-2-NR2 and NcoR-1 peptides were at 200 μM. The co-activator peptide SRC-2-NR2 was derived from the sequence of the co-activator protein SRC-2, and the co-repressor peptide NCoR-1 was derived from the co-repressor protein NCoR-1.

The experiments were performed in 25 mM HEPES pH 7.9, 200 mM NaCl, 5 mm DTT and in the presence of 50 μM dapoxyl-2-amino-ethyl sulfonamide. A 2 μL ligand solution at 2 times the final concentration was dispensed with a micropipette into a 384 well black wall Greiner plate. Then 2 μL of the protein dye solution was dispensed on top of the ligand solution in the 384 well plate. The plates were spun to ensure mixing of the protein-dye and ligand solutions followed by layering of 1 μL of silicone oil to prevent evaporation during heating of the samples. Data were collected on a Thermofluor apparatus and data were analyzed using software that employs a non-linear Marquardt algorithm. Reported results are the average of four experiments.

TABLE 5
Observed Stabilization of PPAR-γ in the Presence of Ligands and the
Co-activator Peptide SRC2-NR2 and Co-Repressor Peptide NCoR-1

| | Ligand | SRC1-NR2 | NCoR-1 |
|---|---|---|---|
| No ligand | N/A | 1.2 | 5.7 |
| Troglitazone | 2.5 | 2.7 | 3.9 |
| Rosiglitazone | 5.6 | 2.1 | 1.3 |
| WY14643 | 1.0 | 1.5 | 5.0 |
| GW9662 | 5.9 | 1.2 | 3.5 |
| BADGE | 1.1 | 1.1 | 5.5 |

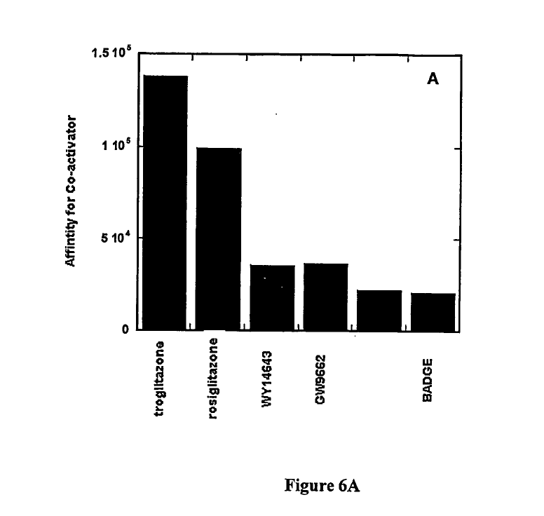

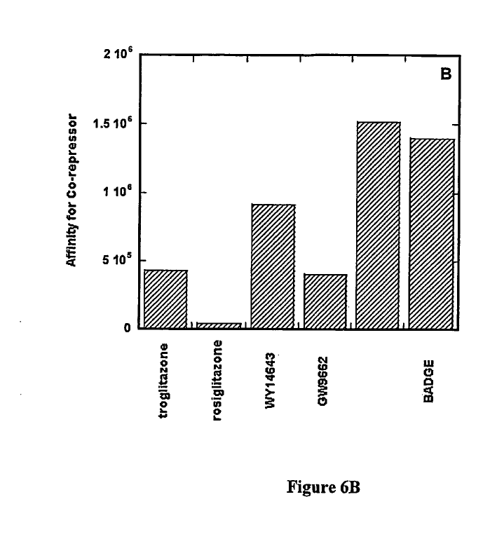

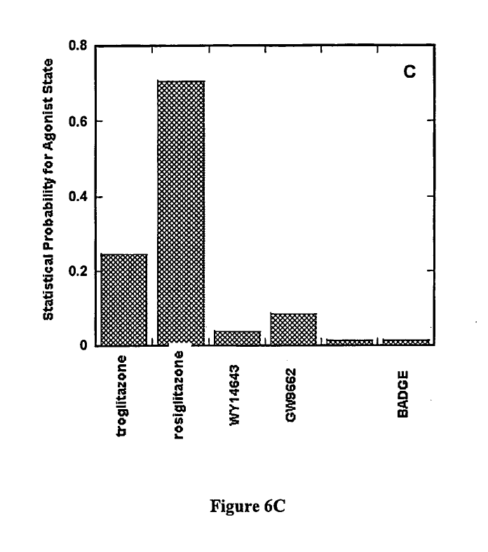

FIG. 6A illustrates calculated binding constants for the co-activator peptide SRC-1NR2 in the absence and in the presence of PPAR-γ ligands. FIG. 6B illustrates calculated binding constants for the co-repressor peptide NCoR-1 in the absence and in the presence of PPAR-γ ligands. FIG. 6C illustrates the calculated statistical probability for the receptor to be in an activated conformation.

From Table 5 and FIGS. 6A, 6B, an 6C we can conclude the following:

a) All ligands affect differentially recruitment of co-activator and co-repressor peptides.

b) PPAR-γ in the presence of troglitazone and rosiglitazone recruits co-activator peptide more efficiently than the free receptor or in the presence of the other ligands (FIG. 6A).

c) No compound recruits co-repressor peptide more efficiently than the ligand free receptor.

d) Troglitazone affects most dramatically both the recruitment of co-activator peptide and rosiglitazone is the second best. Rosiglitazone has the more dramatic effect on co-repressor peptide binding. In other words both ligands, increase binding affinity for the co-activator peptide SRC1-NR2 and decreases the affinity for the co-repressor peptide NCoR-1. From the affinities for the peptides we can calculate the statistical probability of the receptor being in the agonist state for a given ligand (probability of agonist state=(Ka for SRC1-NR2)(Ka for SRC1-NR2+Ka for NcoR1-NR1). The relative, statistical probability in the agonist state of the PPAR-γ:rosiglitazone complex is 70% while for the PPAR-γ:troglitazone complex is 25%. For all other PPAR-γ:ligand complexes in this example is less than 10%.

### Example 6

From FIG. 6C and Table 6, we can observe that the statistical probability for the receptor to be in the agonist state can be a powerful predictor for the efficacy of the ligand. Rosiglitazone is known to be a full agonsist for PPAR-γ while troglitazone is a known partial agonist for PPAR-γ. All other ligands are predicted to be antagonists.

All publications and patents mentioned herein are hereby incorporated by reference in their entireties.

While the foregoing invention has been described in some detail for the purposes of clarity and understanding, it will be appreciated by one skilled in the art from a reading of this disclosure that various changes in form and detail can be made without departing from the true scope of the invention and appended claims.

TABLE 6
Reported Profile of Ligands in Literature and Thermofluor
Prediction Based on Relative Affinities. (partial = partial agonist)

| | | Thermofluor |
|---|---|---|
| Ligand | Literature | Prediction (PPAR-γ) |
| Troglitazone | Partial γ | Partial |
| Rosiglitazone | Full Agonist | Agonist |
| GW9662 | Antagonist γ | Antagonist |
| WY14643 | Partial α, antagonist γ | Antagonist |
| BADGE | Antagonist γ | Antagonist |

1. A method of determining the tissue selectivity of a ligand for a co-regulator dependent target molecule comprising:
(a) providing set of ligands that modify the stability of the target molecule; and
(b) screening one or more ligands of said set for their ability to further modify the stability of the target molecule in the presence of one or more tissue-selective co-regulators for the target molecule; wherein a further modification of stability of the target molecule in the presence of a ligand of said set and a co-regulator of said one or more, tissue-selective co-regulators indicates whether the ligand is an agonist or an antagonist of the target molecule when in the presence of said tissue-selective co-regulator, thereby determining the tissue selectivity of the ligand for the target molecule.

2. The method of claim 1, wherein providing the set of ligands that modify the stability of the target molecule comprises screening one or more of a multiplicity of different ligands for their ability to modify the stability of the target molecule.

3. The method of claim 2, wherein said screening of one or more of a multiplicity of different ligands comprises:
(a) contacting said target molecule with one or more ligands in each of a multiplicity of containers;
(b) treating said target molecules in each of said multiplicity of containers to cause said target molecule to unfold;
(c) measuring in each of said containers a physical change associated with the unfolding of said target molecules;
(d) generating an unfolding curve for said target molecule for each of said containers;
(e) comparing each of said unfolding curves in step (d) to:
(i) each of the other unfolding curves; and/or
(ii) the unfolding curve for said target molecule in the absence of any of said multiplicity of different ligands; and (f) determining whether any of said ligands modifies the stability of said target molecule, wherein a modification in stability is indicated by a change in said unfolding curve.

4. The method of claim 1, wherein said screening step further comprises:
(a) contacting said target molecule and one or more molecules of said set with one or more of said co-regulators in each of a multiplicity of containers;
(b) treating said target molecules in each of said multiplicity of containers to cause said target molecule to unfold;
(c) measuring in each of said containers a physical change associated with the unfolding of said target molecules;
(d) generating an unfolding curve for said target molecule for each of said containers;
(e) comparing each of said unfolding curves in step (d) to:
(i) each of the other unfolding curves; and/or
(ii) the unfolding curve for said target molecule in the absence of (1) any of said ligands of said set and/or (2) said co-regulators; and
(f) determining whether any of said ligands of said set further modifies the stability of said target molecule, wherein a further modification in stability is indicated by a further change in said unfolding curve.

5. The method of claim 1, wherein said one or more co-regulators includes a co-activator and/or a co-repressor.

6. The method of claim 5, wherein one or more ligands of the set further modify the stability of the target molecule in the presence of a co-activator, thereby identifying the ligand as an agonist of the target molecule when in the presence of the co-activator.

7. The method of claim 6, wherein the agonist is a partial agonist;

8. The method of claim 5, wherein one or more molecules of the set further modify the stability of the target molecule in the presence of co-repressor, thereby identifying the ligand as an antagonist of the target molecule when in the presence of the co-repressor.

9. The method of claim 8, wherein the antagonist is a partial agonist.

10. A method of determining the tissue selectivity of a ligand for a co-regulator dependent target molecule comprising:
(a) providing a set of ligands that shift the thermal unfolding curve of the target molecule; and
(b) screening one or more ligands of the set for their ability to further shift the thermal unfolding curve of the target molecule in the presence of one or more tissue-selective co-regulators for the target molecule; wherein a further shift in the thermal unfolding curve of the target molecule in the presence of a ligand of the set and a co-regulator of said one or more tissue-selective co-regulators indicates whether the ligand is an agonist or an antagonist of the target molecule when in the presence of said tissue-selective co-regulator, thereby determining the tissue selectivity of the ligand for the target molecule.

11. The method of claim 10, wherein providing the set of ligands that shift the thermal unfolding curve of the target molecule comprises screening one or more of a multiplicity of different ligands for their ability to shift the unfolding curve of the target molecule.

12. The method of claim 11, wherein said screening of one or more of a multiplicity of different ligands further comprises:
(a) contacting said target molecule with one or more of said multiplicity of different ligands in each of a multiplicity of containers;
(b) heating said target molecules in each of said multiplicity of containers to cause said target molecule to unfold;
(c) measuring in each of said containers a physical change associated with the thermal unfolding of said target molecules resulting from said heating;
(d) generating a thermal unfolding curve for said target molecule as a function of temperature for each of said containers;
(e) comparing each of said unfolding curves in step (d) to:
(i) each of the other thermal unfolding curves; and/or
(ii) the thermal unfolding curve for said target molecule in the absence of any of said multiplicity of different ligands; and
(f) determining whether any of said multiplicity of different ligands shift the thermal unfolding curve of said target molecule.

13. The method of claim 10, wherein said screening step further comprises:
(a) contacting said target molecule and one or more ligands of said set with one or more of said co-regulators in each of a multiplicity of containers;
(b) heating said target molecules in each of said multiplicity of containers to cause said target molecule to unfold;
(c) measuring in each of said containers a physical change associated with the thermal unfolding of said target molecules resulting from said heating;
(d) generating a thermal unfolding curve for said target molecule as a function of temperature for each of said containers;
(e) comparing each of said thermal unfolding curves in step (d) to:
(i) each of the other thermal unfolding curves; and/or
(ii) the thermal unfolding curve for said target molecule in the absence of (1) any of said ligands of said set and/or (2) said co-regulators; and
(f) determining whether any of said molecules of said set further shifts the thermal unfolding curve of said target molecule.

14. The method of claim 10, wherein said one or more co-regulators includes a co-activator and/or a co-repressor.

15. The method of claim 10, wherein one or more ligands of the set further modify the stability of the target molecule in the presence of a co-activator, thereby identifying the ligand as an agonist of the target molecule when in the presence of the co-activator.

16. The method of claim 15, wherein the agonist is a partial agonist.

17. The method of claim 10, wherein one or more ligands of the set further modify the stability of the target molecule in the presence of a co-repressor, thereby identifying the ligand as an antagonist of the target molecule when in the presence of the co-repressor.

18. The method of claim 17, wherein the antagonist is a partial agonist.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A method of determining the tissue selectivity of a ligand for a co-regulator dependent target molecule having an unknown function comprising:
(a) providing a set of ligands that modify the thermal unfolding curve of a target molecule having an unknown function, wherein said set of ligands modify the thermal unfolding curve of receptors which share biological function; and
(b) screening one or more ligands of said set for their ability to further modify the thermal unfolding curve of the target molecule in the presence of one or more co-regulators; wherein a further modification of the thermal unfolding curve of the target molecule in the presence of a ligand of said set and a co-regulator of said one or more co-regulators indicates whether the molecule is an agonist or an antagonist of the target molecule when in the presence of said co-regulator.

40. The method of claim 39, wherein providing the set of ligands that modify the thermal unfolding curve of the target molecule comprises screening one or more panels of ligands which modify the thermal unfolding curve of receptors which share biological function for their ability to modify the thermal unfolding curve of the target molecule.

41. The method of claim 39, wherein the ligand is a partial agonist of the target molecule when in the presence of a co-activator.

42. The method of claim 39, wherein the ligand is a partial agonist of the target molecule when in the presence of a co-repressor.

43. A method of determining the tissue selectivity of a ligand for a co-regulator dependent target molecule having an unknown function comprising:
(a) providing a set of ligands that modify the stability of a target molecule having an unknown function, wherein said set of ligands modify the stability of receptors which share biological function; and
(b) screening one or more ligands of said set for their ability to further modify the stability of the target molecule in the presence of one or more co-regulators; wherein a further modification of the stability of the target molecule in the presence of a ligand of said set and a co-regulator of said one or more co-regulators indicates whether the molecule is an agonist or an antagonist of the target molecule when in the presence of said co-regulator.

44. The method of claim 43, wherein providing, the set of molecules that modify the stability of the target molecule comprises screening one or more panels of ligands which modify the stability of receptors which share biological function for their ability to modify the stability of the target molecule.

45. The method of claim 43, wherein the ligand is a partial agonist of the target molecule when in the presence of a co-activator.

46. The method of claim 43, wherein the ligand is a partial agonist of the target molecule when in the presence of a co-repressor.

47. The method of claim 1, wherein the target molecule is selected from androgen receptor, glucocorticoid receptor, estrogen receptor, progesterone receptor, GPCR,NF-KB, steroid receptor co-activator (src), and Jac.

48. The method of claim 1, wherein the target molecule is a nuclear receptor.

49. The method of claim 1, wherein the target molecule is a G-protein coupled receptor.

50. The method of claim 1, wherein the target molecule is an estrogen receptor.

51. The method of claim 1, wherein the target molecule is ER-a.

52. The method of claim 1, wherein the target molecule is ER-.

53. The method of claim 1, wherein the target molecule is PPAR-Y.

54. The method of claim 1, wherein the target molecule is a tyrosine kinase.

55. The method of claim 1, wherein the target molecule is NF-xB.

56. The method of claim 1, wherein one or more of the co-regulators are selected from SRC-1, SRC-2, and SRC-3.

57. The method of claim 1, wherein the co-regulator is selected from Gsa, Gia, Gta, Gqa, Goa, Gqa, IlcB, SH2 and SOCS.

58. The method of claim 1, wherein the ligand is a steroid.

59. The method of claim 1, wherein the ligand is a non-steroidal ligand.
